(19) United States
(12) Patent Application Publication
(10) Pub. No.: US 2026/0248481 A1
(43) Pub. Date: Aug. 27, 2026

(54) SYSTEMS AND METHODS FOR SCOLIOSIS ASSESSMENT AND PROGRESSION PREDICTION

(71) Applicants: Edmond Hok Ming LOU, Edmonton (CA); Marek Zenon REFORMAT, Edmonton (CA); Carmen Hoi-wen LEUNG, Edmonton (CA); Weiying CHEN, Edmonton (CA)

(72) Inventors: Edmond Hok Ming LOU, Edmonton (CA); Marek Zenon REFORMAT, Edmonton (CA); Carmen Hoi-wen LEUNG, Edmonton (CA); Weiying CHEN, Edmonton (CA)

(21) Appl. No.: 19/548,466

(22) Filed: Feb. 24, 2026

Related U.S. Application Data
(60) Provisional application No. 63/764,334, filed on Feb. 27, 2025.

(51) Int. Cl. A61B 8/08; A61B 8/00

(52) U.S. Cl. CPC A61B 8/0875; A61B 8/466; A61B 8/468; A61B 8/483; A61B 8/5223; A61B 8/5246

(57) ABSTRACT

Systems and methods are discussed for capturing ultrasound images of a person's spine, in particular, a stereo camera integrated with a handheld wireless ultrasound scanner, which can simultaneously capture surface topography, internal spinal alignment, and bone quality information.

### CROSS-REFERENCE TO RELATED APPLICATIONS

This application claims priority of United States Provisional Patent App. No. 63/764,334 filed Feb. 27, 2025, which is incorporated by reference into this application in its entirety.

### TECHNICAL FIELD

The present disclosure is related to systems and methods for capturing ultrasound images of a person's spine, in particular, a stereo camera integrated with a handheld wireless ultrasound scanner, which can simultaneously capture surface topography, internal spinal alignment, and bone quality information.

### BACKGROUND

Adolescent idiopathic scoliosis (“AIS”) is a three-dimensional (“3D”) abnormal spinal curvature with vertebral rotation and may have sagittal abnormality. It affects 3% of adolescents; most patients are female adolescents [1]. The primary parameters used to quantify the severity of scoliosis are the Cobb angle, vertebral rotation, kyphotic angle, and lordotic angle measured from posteroanterior (“PA”) and lateral (“LAT”) radiographs.

Adolescents with a progressive or moderate size of spinal curvature (Cobb angle ≥25° and <45°) are recommended non-surgical treatment, either exercise or brace treatment or a combination of both. Invasive surgery is recommended when the Cobb angle is greater than 45°. Unfortunately, there is no reliable method to predict which patients will develop worsening curves. Subjective judgment from the treating clinicians is the current standard.

Routine radiographs are required to diagnose, monitor, and determine the treatment option as often as 4 times per year in patients at high risk of progression. Taking radiographs exposes patients to ionizing radiation, which increases the risk of cancer and is not desirable for children and adolescents.

In a two-dimensional (“2D”) PA view radiograph, the Cobb angle is the standard to diagnose, guide treatment decisions, monitor progression, and quantify treatment outcomes. The Cobb method [2], a summation of the most tilted angles of the superior endplate vertebra and inferior endplate vertebra, is the gold standard for measuring the coronal curvature, called the Cobb angle.

Vertebral rotation is another feature used to evaluate scoliosis and influences treatment planning. Landmark identification techniques [3-8] have been developed to quantify the severity of vertebral rotation. However, it is a time-consuming process, and most of the clinics do not perform quantitative measurements. Among many methods, Stokes'method [7] is the most used to provide quantitative value. It combines the position of the pedicles relative to the center of the vertebral body with an available vertebral model to obtain width-to-depth ratio on a specific vertebral level to calculate the rotation of the vertebra. However, this merely estimates the rotation, as the measurement is performed on a 2D PA radiograph.

The kyphotic and lordotic angles, taken from a LAT radiograph, are commonly measured at the initial visit to evaluate potential deformity in the sagittal plane, the risk of progression, and the appropriateness of treatment modalities. The Cobb method, which measures the angle between the superior endplate of the top thoracic vertebra (“T1” level) and the inferior endplate of the last thoracic vertebra (“T12”), is the most used method to determine the kyphotic angle. Although widely used, it offers limited reproducibility and accuracy due to difficulty identifying the superior endplate at the upper thoracic region. Lordotic angle uses a similar measurement approach, except it is measured on the superior endplate of the first lumbar vertebra (“L1” level) and the inferior endplate of the last lumbar vertebra (“L5”) or the superior endplate of S1 vertebra.

Ultrasound imaging is a non-invasive and non-destructive method that can capture the 3D spinal bone structure when the ultrasound systems have the position and orientation information for each acquired B-mode image. Research has been conducted and validated that coronal curvature angle (Cobb angle), vertebral rotation, and kyphotic angle can be measured reliably on ultrasound images manually [9-12]. However, the reported ultrasound systems are bulky and take up a lot of space. Recently, portable 3D ultrasound systems [13-14] have been available for scoliosis applications, but they are neither truly wireless nor provide external surface images.

Surface topography (“ST”) is another non-invasive imaging method that captures a 3D map of the torso using non-contact optical systems. It is used as an alternative assessment method for scoliosis. It analyzes 3D features of the torso without any harmful side effects. Technological advances in scanning have led to scanning accuracy and speed of reconstruction. However, the surface information alone cannot report the true internal spinal alignment.

From the literature, it has been reported that osteopenia is a prognostic factor for children with scoliosis. It was found that dual-energy x-ray absorptiometry (“DXA”) could be used to measure bone mineral density (“BMD”) at the femoral neck to predict curve progression in children. However, DXA involves ionizing radiation and is undesirable to children. The application of quantitative ultrasound (“QUS”) has been shown to reliably assess bone mineral status in children. However, the QUS method does not assess bone quality in the spine region.

Convolutional neural network (“CNN”) is a subset of artificial intelligence used primarily to classify images, cluster them by similarity, and perform object recognition within images. The objectives of CNNs are to automatically learn new features from databases and to generalize their responses to circumstances not encountered during the learning phase. This technology can be used to improve measurement reliability and provide higher accuracy in automatic measurement. Machine learning algorithms have already been developed to measure the Cobb angle on PA radiographs. This can be applied to ultrasound and surface topography images to measure spinal curvature automatically. In addition, utilizing artificial intelligence can predict the progression of scoliosis

It is, therefore, desirable to provide systems and methods for capturing ultrasound images of a person's spine that overcomes the shortcomings of the prior art.

### SUMMARY

In some embodiments, a stereo camera integrated with a handheld wireless ultrasound scanner can be used to simultaneously capture surface topography, internal spinal alignment, and bone quality information. The stereo camera can not only capture the surface topography of patients'back images but can also provide spatial information to allow ultrasound images to reconstruct a 3D spine. The bone quality of the lowest lumbar vertebra will be acquired as additional information. An artificial intelligence image analyzer is provided to display and extract the 3D topography and ultrasound spine images and measurements, which can also be used to predict the risk of curve progression and the future curvature of scoliosis.

By way of introduction, the embodiments described below include a unique portable wireless ultrasound scanner with an integrated stereo camera and method to capture ultrasound and back surface topography images simultaneously. After processing, the 3D ultrasound spinal image and 3D back-only surface topography image can be displayed. These images can be used to diagnose, monitor, and assess the severity of scoliosis separately or in combination. In addition, the acquired information can provide a method to estimate the risk of progression of scoliosis and predict future spinal curvature changes.

In some embodiments, a system can be provided for screening, diagnosing, monitoring, and assessing the severity of scoliosis in a radiation-free and non-invasive manner. The system can comprise a handheld device including a portable wireless ultrasound scanner, a stereo camera, a mini-personal general purpose computer (“PC”), a battery pack, and a processing laptop/desktop. The ultrasound scanner can capture the internal spinal structure by obtaining the B-mode spinal axial images and steams to the laptop/desktop wirelessly. The stereo camera can capture the back's surface topography and provides the position and orientation of the ultrasound scanner. An inertial measurement unit (“IMU”), which can be embedded inside the stereo camera, can comprise a 3-axis accelerometer, a 3-axis gyroscope, and a 3-axis magnetometer. By using a developed algorithm that can combine the surface images at different times and the IMU information, a 6-degree-of-freedom pose information can be obtained accurately. The ultrasound scanner can stream the ultrasound images and raw data information wirelessly via WiFi Direct® to the processing laptop or desktop. On the other hand, the stereo camera with the built-in IMU, which connects to a mini-PC also sends pose and image information to the processing PC via either wired or wirelessly. The streamed ultrasound images can then be constructed into 3D and displayed on the laptop/desktop. The ultrasound scanner and the stereo camera can also be operated independently.

In some embodiments, the system can also comprise a foot pedal with multiple buttons to control the ultrasound and stereo camera acquisition wirelessly.

In some embodiments, the functions of the processing laptop/desktop can process ultrasound images, camera images, position and orientation information, parameter extraction, and prediction models.

In some embodiments, the system can include software that can reconstruct 3D ultrasound spine image and 3D surface topography of a patient's back image in the processing PC using the ultrasound spinal axial images, position and orientation information from the IMU of the stereo camera, and the camera-captured images. Since the stereo camera is close to the body, approximately 25 to 35 cm away, only a section of the back image can be captured at each position. Therefore, the position and orientation data can provide the necessary information to stitch the ultrasound axial and stereo-camera images to produce full 3D internal spine and external surface reconstructed images. In some embodiments, the software can automatically conduct image processing methods of de-noising, image filtering, and contrast enhancement to improve visualization of the final images.

In some embodiments, a graphical user interface can be provided to visualize the rendered 3D spinal reconstruction and/or surface topography images. The ability to manipulate the views of both 2D and 3D images can be provided, including translation, rotation, magnification, and cropping. The ability to view coronal and sagittal projections, along with a view of a selectable transverse image, can be provided for the ultrasound images. The ability to overlay the ultrasound coronal projection on the back surface can be provided to garner a fuller understanding of the internal and external spinal alignment. The module can also allow the operator to annotate various spinal landmarks on these different views to measure different scoliotic parameters. Once the required landmarks are annotated, the module can automatically measure the parameter and displays the value for the operator.

In some embodiments, an automatic spinal landmark annotation module can be provided to facilitate scoliotic parameters measurements. This module can be based on a CNN that aims to segment the landmarks, laminae, and spinous processes of the ultrasound data. This automation module can be combined with the graphical user interface, meaning the operator can acquire starting automatic annotations first and then manually adjust the placement of the predictions, if necessary, before obtaining the values of the scoliotic parameters.

In some embodiments, an automatic surface topography parameters extraction module can be provided that uses image processing to automatically calculate various surface topography parameters, including shoulder angle differences, shoulder height differences, scapular asymmetry, lateral displacement, waist asymmetry, pelvis asymmetry, waist crease, trunk rotation, sagittal balance, etc.

In some embodiments, scoliosis curve progression prediction models for patients who are under observation, brace treatment, or exercise treatment, based on ultrasound parameters, including Cobb angle, vertebral rotation, kyphotic angle, lordotic angle, spine flexibility, bone quality, and bone age plus the surface topography parameters, including shoulder angle differences, shoulder height differences, scapular asymmetry, lateral displacement, waist asymmetry, pelvis asymmetry, waist crease, trunk rotation, sagittal balance, and other patients'demographical information can be provided. The prediction models can include the probability of estimating the scanned subject's curve severity, including the progress or not progress, and the curve severity angle before the subject's next clinical visit for 6 months to 12 months. The prediction model can also predict the final scoliotic curve outcomes after the treatment.

The systems and methods described herein can advantageously provide unlimited frequency of usage for screening, diagnosing, monitoring, and evaluating scoliosis, as well as predicting the outcomes of scoliosis at different stages. In some embodiments, the systems and methods described herein can be non-invasive and safe to use. In some embodiments, the systems and methods described herein can be cost-effective and can be operated anytime and anywhere without requiring a specific clinical environment.

In some embodiments, a method of calculating the position and orientation of the ultrasound transducer can be derived from: using the time stamps of the captured stereo-images; the vision method which calculates the camera's motion based on the image information; and the estimated position change based on the IMU. Combining the three pieces of information can provide an accurate movement of the stereo camera, providing accurate position and orientation of the ultrasound transducer during scanning.

In some embodiments, a method can be provided to reconstruct an ultrasound volume using the position and orientation data obtained from the stereo camera.

In some embodiments, a display module can be provided for aligning and displaying the ultrasound and surface topography reconstructions together.

In some embodiments, a manual annotation module can be provided for marking ultrasound and surface topography features for measuring parameters describing the severity of scoliosis.

In some embodiments, a convolutional neural network can be provided to automatically predict the centers of laminae on ultrasound reconstruction to facilitate quicker measurement of scoliotic parameters.

In some embodiments, a method can be provided for measuring the spinal flexibility of a scanned subject using both ultrasound and surface topography data.

In some embodiments, a method based on machine learning can be provided for automatically measuring ultrasound various ultrasound parameters, including the Cobb angle, kyphotic angle, lordotic angle, vertebral rotation, and the plane of maximum deformity.

In some embodiments, a method based on machine learning can be provided for automatically displays the estimated 3D vertebrae to display the spine column visually.

In some embodiments, a method based on machine learning can be provided for predicting whether the scanned subject's curve will progress before the next clinical visit six months in the future using the measured ultrasound and surface topography parameters, coupled with demographic parameters, using a random forest, neural network, or gradient-boosted trees.

In some embodiments, a method based on machine learning can be provided for generating a video for predicting how the scoliotic subject's curve will progress for the next clinical visit, given the deformity severity parameters.

Broadly stated, in some embodiments, an imaging system and method can be provided for screening, diagnosing, monitoring, and assessing treatment of a scoliotic patient, the system comprising: an ultrasound scanner for capturing internal spinal structural changes of the patient by acquiring a stream of axial B-mode images thereof (“ultrasound images”); a stereo-camera to capture images of a back surface topography of the patient (“stereo-camera images”); an inertia measurement unit (“IMU”) disposed inside the stereo-camera to produce position and orientation information of the stereo-camera (“IMU data”); a mini-personal computer (“PC”) operatively coupled to the stereo-camera for storing the IMU data and for storing the stereo-camera images; a battery for providing electrical power for the mini-PC and for the stereo-camera; a central processing unit comprising a display, and at least one software module comprising software code segments configured for: reconstructing at least one spine and back surface image from the ultrasound images and the stereo-camera images, displaying and manipulating the at least one spine and back surface image in three dimensions (“3D”), manually annotating spinal and surface landmarks on the at least one spine and back surface image, automatically annotating the spinal and surface landmarks using machine learning, measuring scoliotic and surface topography parameters based on the annotated spinal and surface landmarks, and predicting curve progression using machine learning; and a wireless control pedal for controlling the operation of the system.

Broadly stated, in some embodiments, the system and method can wirelessly stream the ultrasound images to the central processing unit with a time stamp indicated on each image thereof.

Broadly stated, in some embodiments, the mini-PC can be configured to transfer the IMU data and the stereo-camera images with at least one time stamp associated with each thereof to the central processing unit after an ultrasound scan of the patient has been completed.

Broadly stated, in some embodiments, the at least one software module can be configured to synchronize the IMU data and the stereo-camera images at the at least one time stamp.

Broadly stated, in some embodiments, the software code segments can be configured to align the ultrasound images with the IMU data and the stereo-camera images at the at least one time stamp.

Broadly stated, in some embodiments, the software code segments can be configured to reconstruct the at least one spine and back surface image in 3D based on the IMU data and the stereo-camera images.

Broadly stated, in some embodiments, the system and method can be configured to synchronize the ultrasound data images with the stereo-camera images.

Broadly stated, in some embodiments, the system and method can be configured to calculate position and orientation data of the ultrasound scanner transducer using the IMU data and stereo-camera images.

### BRIEF DESCRIPTION OF THE DRAWINGS

FIG. 1 is a block diagram depicting one embodiment of a hand-held ultrasound stereo-camera scanner system.

FIG. 2 has perspective and side elevation views depicting the hand-held stereo camera of FIG. 1.

FIG. 3 depicts a set of ultrasound images in 3 different views (coronal, sagittal, and axial).

FIG. 4 depicts a back surface topography image with parameters indication.

FIG. 5 depicts images of a patient including a coronal ultrasound image, a surface topography back image, an X-ray image, sagittal ultrasound and sagittal topography images from the left, sagittal topography image from the right, and a sagittal X-ray.

FIG. 6 depicts a coronal surface topography image, a coronal image of ultrasound spine, and an overlaid of external surface topography on top of the ultrasound spine of the scanned subject.

FIG. 7 is a flowchart depicting one embodiment of the overall operation of the systems and methods described herein, including scanning, reconstruction, annotation, parameter measurement, and progression prediction.

FIG. 8 depicts a set of different scoliotic measurements performed on the various ultrasound views, including the Cobb angle on the coronal view, the kyphotic angle on the sagittal view, and vertebral rotation on the axial view.

FIG. 9 depicts an example of a reflection coefficient measurement performed on the coronal projection of the ultrasound reconstruction to estimate bone quality.

FIG. 10 depicts one embodiment of a concept of the plane of maximum deformity.

FIG. 11 depicts an example of a spine flexibility measurement performed on (a) the standing coronal ultrasound image, (b) bending to the left ultrasound image, and (c) bending to the right ultrasound image.

FIG. 12 is a perspective view depicting an alternate embodiment of the hand-held stereo camera of FIG. 2.

### DETAILED DESCRIPTION OF EMBODIMENTS

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment can also be included in other embodiments but is not necessarily included. Thus, the present technology can include a variety of combinations and/or integrations of the embodiments described herein.

The presently disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

While the following terms used herein are believed to be well understood by one of ordinary skill in the art, definitions are set forth to facilitate explanation of the presently disclosed subject matter.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments +/−50%, in some embodiments +/−40%, in some embodiments +/−30%, in some embodiments +/−20%, in some embodiments +/−10%, in some embodiments +/−5%, in some embodiments +/−1%, in some embodiments +/−0.5%, and in some embodiments +/−0.1% from the specified amount, as such variations are appropriate to perform the disclosed method.

Alternatively, the terms “about” or “approximately” can mean within an acceptable error range for the particular value as determined by one of ordinary skill in the art, which will depend in part on how the value is measured or determined, i.e., the limitations of the measurement system. For example, “about” can mean within 3, or more than 3, standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise indicated, all numbers expressing quantities, properties, and so forth used in the specification and claims are to be understood as being modified in all instances by the term “about”. And so, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10and 15 are disclosed, then 11, 12, 13, and 14 are also disclosed.

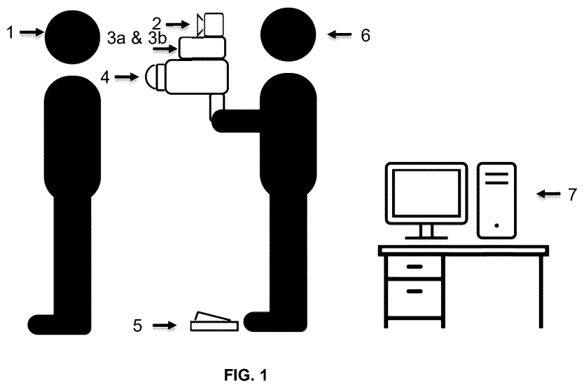

Referring to FIG. 1, one embodiment of a handheld stereo-camera ultrasound system to capture surface topography and internal spinal alignment simultaneously for scoliosis assessment is shown. In some embodiments, the system can scan a subject with scoliosis 1, comprising of a stereo-camera 2, a mini-PC 3a, a battery 3b, a wireless ultrasound scanner 4, and a wireless pedal system for hands-free operation 5. When an operator 6 performs the scanning, the ultrasound images and data produced by the system can be streamed to a central processing unit 7 (computer, laptop, or tablet) wirelessly. The stereo-camera images can be sent to the mini-PC 3a first and then transferred to the processing unit 7 via wire or wireless to reconstruct both ultrasound and surface topography images. Parameter measurements and scoliosis prediction can then be processed in the processing unit 7.

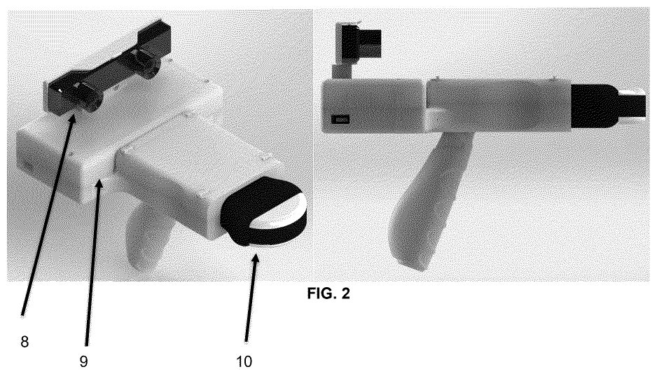

FIG. 2 shows the 3D handheld stereo-camera ultrasound device in 2 different views. In some embodiments, the device can comprise the stereo camera 8, the mini-PC with battery 9, and an ultrasound scanner 10 in a custom design enclosure.

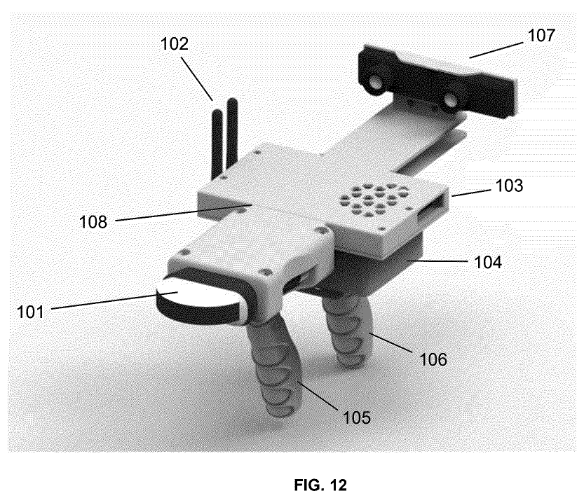

Referring to FIG. 12, an alternative embodiment of the handheld stereo-camera ultrasound device of FIG. 2 is shown. In some embodiments, the handheld stereo-camera ultrasound device can capture surface topography and internal spinal alignment simultaneously for scoliosis assessment. In some embodiments, the device can be separated into two independent units, releasably and operatively connected at location 108. In some embodiments, one of the two independent units can comprise portable ultrasound scanner 101 having handle 105. In some embodiments, the other of the two independent units can comprise stereo camera 107 operatively coupled to mini-PC 104 and battery 103, the combination of which can comprise handle 106. In some embodiments, mini-PC 104 can be operatively coupled to Wi-Fi® antenna 102. In some embodiments, the ability to separate the device into the two independent units is to increase its portability. When the device is separated into the two independent units, each unit can be held or supported by handles 105 and 106.

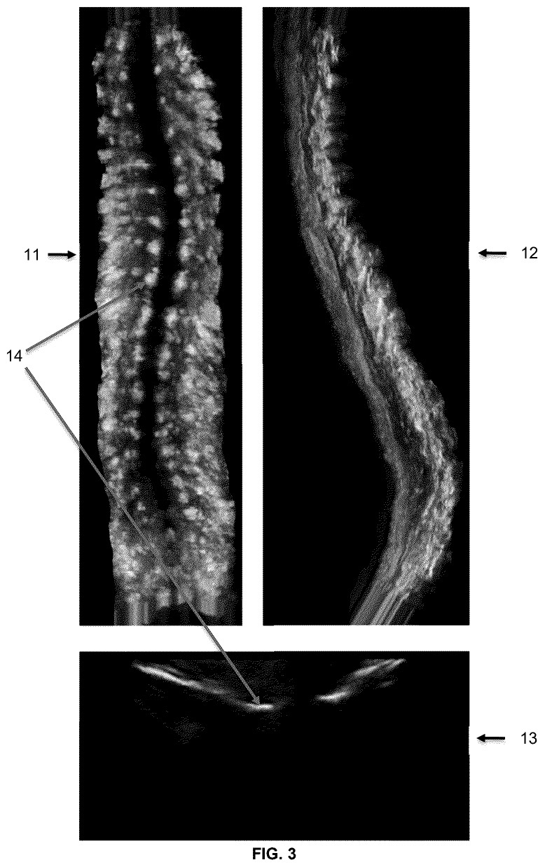

FIG. 3 shows the three different views, coronal view 11, sagittal view 12, and transverse view 13, of projected ultrasound images based on the 3D spinal reconstruction volume generated by a scan of a subject's spine. In some embodiments, this volume can be reconstructed using the spinal axial images combined with the position and orientation information generated by the stereo-camera. The strong reflection signals (white spots) close to the center on the coronal view are the laminae 14. The sagittal projection can be used to visualize the kyphotic and lordotic angles. Individual transverse images can be used to confirm the centers of laminae and measure the axial vertebral rotation.

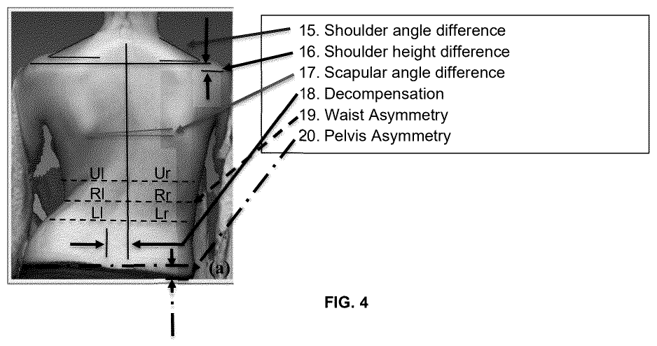

FIG. 4 shows the back only surface topography from a scoliotic patient. Six parameters are shown on the image. The shoulder angle difference 15 is the angle difference between the left and right shoulder angles. The shoulder height difference 16 is the height difference between the left and right shoulders. The scapular angle difference 17 is the angle calculated based on the tip of left and right scapular points relative to horizontal. Decompensation 18 is the horizontal side shift based on the mid-point of the neck and the mid-point of the pelvis. Waist asymmetry 19 can be calculated based on six distances, including Ur (upper right), Ul (upper left), Rr (reference right), Rl (reference left), Lr (lower right), and Ll (lower left). The vertical reference line can be drawn upward from the midpoint of the posterior superior iliac spine (PSIS) which is the posterior edge of the iliac crest. The horizontal reference line was the minimum distance at the waist region. The upper and lower horizontal lines were drawn at 10% of the trunk length above and below the horizontal reference line. The pelvis asymmetry 20 is the vertical distance between the left and right pelvis points.

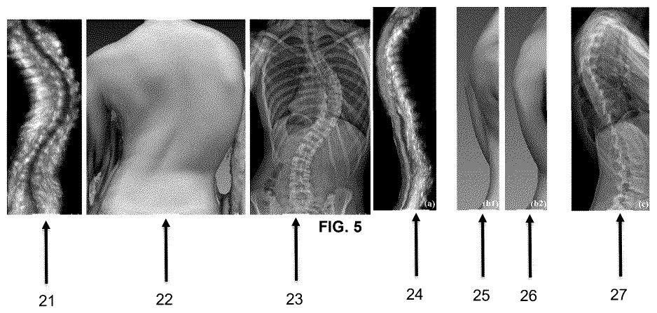

FIG. 5 shows the coronal ultrasound image 21, the patient's back image 22 the coronal X-ray image 23, the sagittal ultrasound image 24, the surface topography from the left side 25 and the right side 26, and the sagittal X-ray image 27.

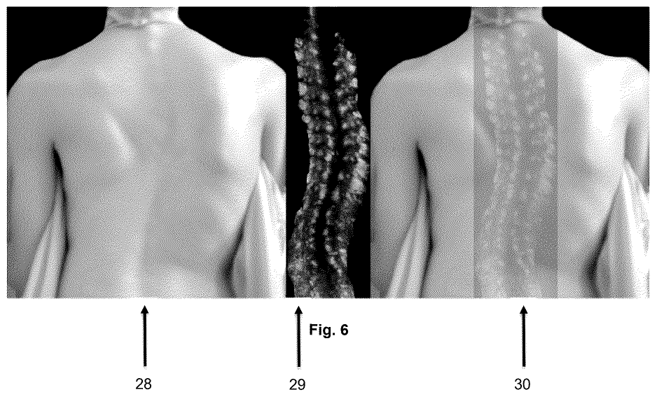

FIG. 6 shows an example of surface topography data 28 combined with the corresponding ultrasound maximum coronal projection 29. In some embodiments, with the ultrasound and stereo-camera data synchronized during scanning, an image overlay of both streams of data 30 can be generated to display both the external and internal alignment of the spine.

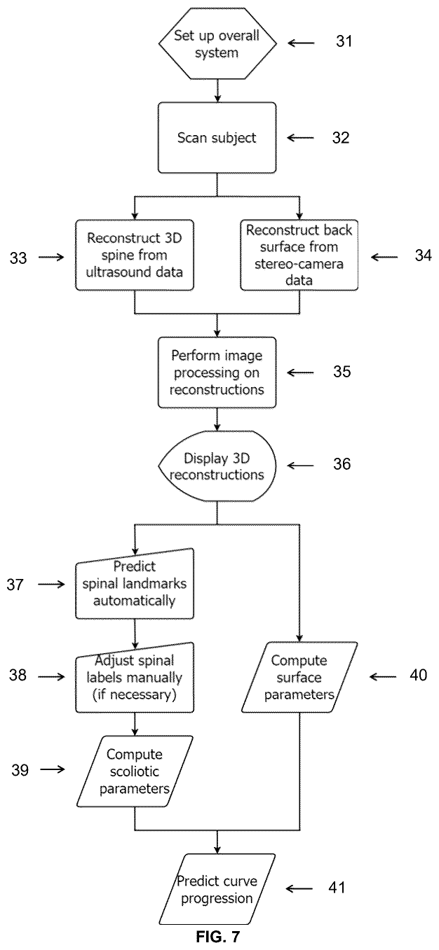

FIG. 7 shows the flowchart of the overall procedure using the preferred ultrasound transducer and stereo-camera system. In some embodiments, the components shown from FIG. 1 can be initially set up, labelled as 31, by connecting the ultrasound transducer 4 to the central processing unit 7 wirelessly using Bluetooth and Wi-Fi direct and the stereo-camera 2 to the mini-PC 3a through wire. The operator can determine the mode of operation (surface topography only or ultrasound and surface topography combined) using a set of pedals 5. Live feedback on the active mode of operation can be provided on the central processing unit 7. The operator can modify various ultrasound scanning parameters, including but not limited to frequency and gain, on the central processing unit 7 to optimize image quality. Once set up, the subject is asked to position himself/herself according to the operator's instructions. If the current mode includes ultrasound scanning, ultrasound gel can be applied to the subject's back before scanning and markers are placed onto the subject's back so that the stereo-camera 2 can determine the position and orientation of the image throughout the scan.

The subject's back and spine can then be scanned, labelled as 32, with the combined transducer and stereo-camera. The subject 1 is asked to be as still as possible and hold their breath during this procedure. The operator 6 can place the device, at which the ultrasound transducer can contact firmly on the subject's back at the uppermost thoracic vertebra T1 and presses the pedal 5 to start data acquisition. The operator 6 can then move the device downwards steadily, following the contour of the spinal curvature, until the lowermost lumbar vertebra L5 is reached. During the scan, the ultrasound transducer 4 can touch the subject's back and the stereo-camera 2 can be mounted such that its lens is 25 to 35 cm away from the subject's back. To signal the end of the scan, the operator 6 can step on the pedal 5 or press a key on the central processing unit 7 or press a button on the ultrasound scanner 4. All data related to the ultrasound transducer 4, including ultrasound B-mode images and radio frequency acoustic images, can be streamed to the central processing unit 7 during the scan. The images acquired by the stereo-camera 2 can be streamed to the min-PC 3a during the scan since the stereo camera and the ultrasound cannot stream the data to the central processing unit 7 simultaneously through Wi-Fi Direct. Once the scan is complete, the mini-PC 3a can connect to the central processing unit 7 to transfer the stereo-images so that they can be combined with the ultrasound data. A software module can align the timestamps of the two streams of images to combine and reconstruct the data, generating a 3D ultrasound spinal reconstruction, labelled as 33, and/or surface of the subject's back, labelled as 34, depending on the mode of operation. Further image processing, labelled as 35, can be conducted automatically to optimize the rendering of the 3D reconstruction(s), labelled as 36. A software module can then display the reconstructed image(s) on the central processing unit screen 7 so that the operator 6 can view and evaluate the subject's deformity. The module allows the operator 6 to manipulate the views by translating and rotating the reconstruction(s) in all three axes, as well as zooming in and out. A manual contrast adjustment module is also available to improve spinal landmark prominence.

In some embodiments, a software module for automatic prediction of spinal landmarks can be provided, labelled as 37. A CNN can be trained to predict the positions of the centers of lamina. A software module for manually adjusting spinal landmarks can be provided so that the operator can adjust any landmarks that were placed incorrectly by the CNNs, labelled as 38. The software module can then calculate various internal scoliotic parameters, labelled as 39, using the identified landmarks. These can include the Cobb angle, the plane of maximum deformity, the kyphotic angle, the lordotic angle, axial vertebral rotation, the reflection coefficient, and spinal flexibility.

In some embodiments, a software module for automatically calculating surface topography parameters, labelled as 40, can be provided. Image processing can be performed on the stereo-image data to determine the 6 parameters shown in FIG. 4. In some embodiments, a machine learning model to predict the risk of curve progression, labelled as 41, can also be provided to aid clinicians with treatment management. It can use the measured parameters, coupled with input demographic parameters such as age and gender, as inputs and can then output the probability that the subject's curvature severity will worsen significantly before the next clinical visit. With this, the machine learning model can also generate a video that predicts how the scanned subject's curve will progress for the next clinical visit.

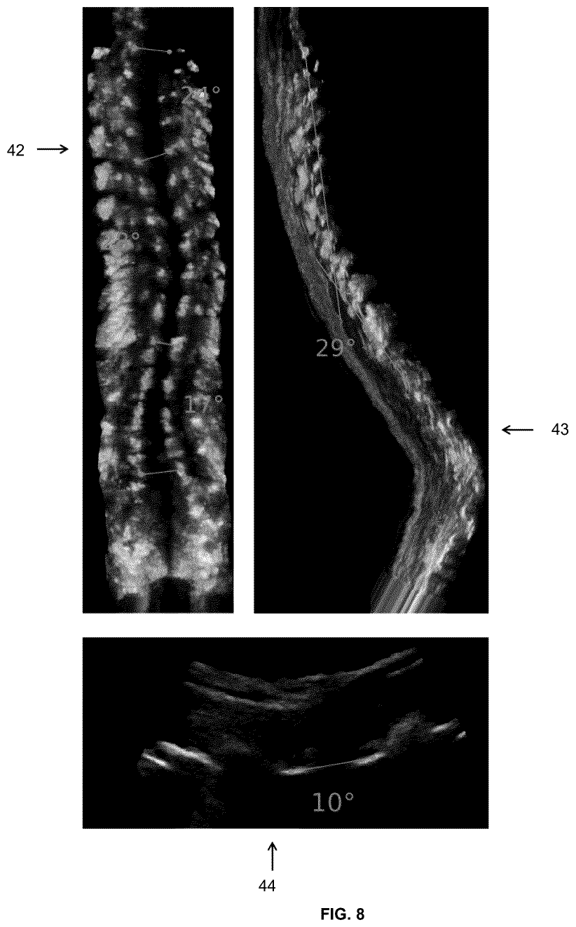

FIG. 8 shows examples of scoliotic measurements performed on the ultrasound reconstruction. The maximum coronal projection, labelled as 42, illustrates the Cobb angle, where the dots indicate the centers of laminae. Lines are drawn between each pair of laminae, and the angle of each line relative to the horizontal is calculated. The Cobb angles can then be calculated as the sums of the angles of the most opposingly tilted lines. The maximum sagittal projection, labelled as 43, illustrates the kyphotic angle, where the dots indicate the centroids of the left-right lamina pairs for the T1, T2, T11, and T12 vertebrae. The angles of the lines joining the T1-T2 and T11-T12 centroids can be determined, and the sum of the angles of these two lines is the kyphotic angle. The axial slice, labelled as 44, illustrates the vertebral rotation. The vertebral rotation can be measured as the angle formed when joining the left and right centers of the laminae, indicated by the dots.

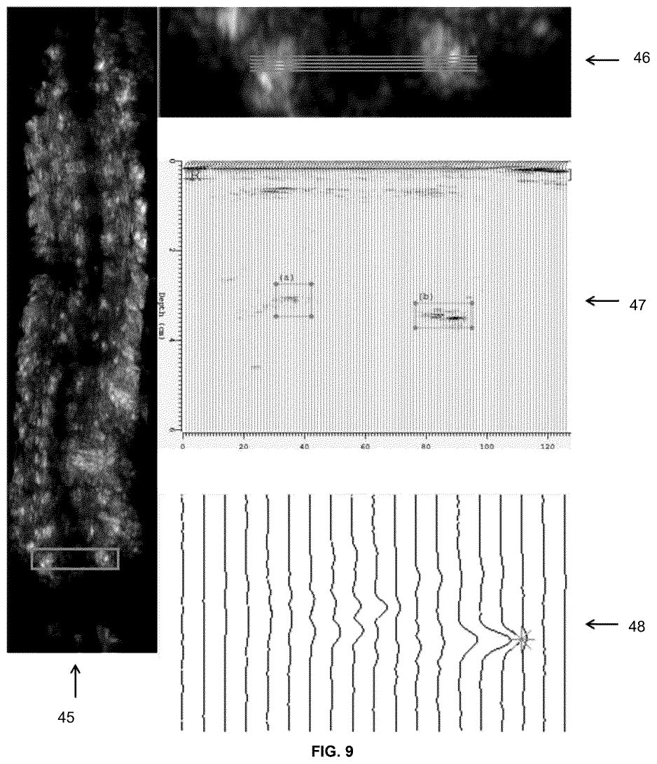

FIG. 9 shows an example of a reflection coefficient measurement performed on the ultrasound reconstruction. The maximum coronal projection image, labelled as 45, highlights the L5 vertebra, which can be used to derive the reflection coefficient, a proxy for bone quality. The magnified L5 vertebra, labelled as 46, illustrates the five frames, indicated by the five horizontal lines, can be used for reflection coefficient measurements. The radio frequency data visualization, labelled as 47, shows the corresponding reflection signals for an L5 frame with the left and right laminae boxed. The raw ultrasound signals for the right lamina, labelled as 48, illustrate the thickness of the soft tissue from the skin surface to the lamina reflection point, which is used to derive the final reflection coefficient value.

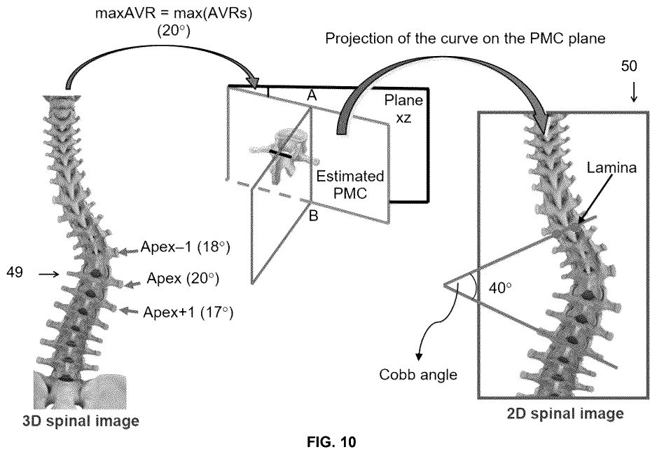

FIG. 10 shows the concept of the plane of maximum deformity. Because 3D data can be captured in the ultrasound scan, individual vertebral rotations can be directly measured. The plane of maximum deformity is the rotated coronal plane in which the vertebra with the highest rotation (typically at the apex of the most severe curve, labelled as 49) has a 0° rotation. Measuring the Cobb angle in the plane of maximum deformity, labelled as 50, can be typically more severe than in the standard coronal projection, and so determining this plane allows the operator to grasp the 3D nature of the spinal deformity fully.

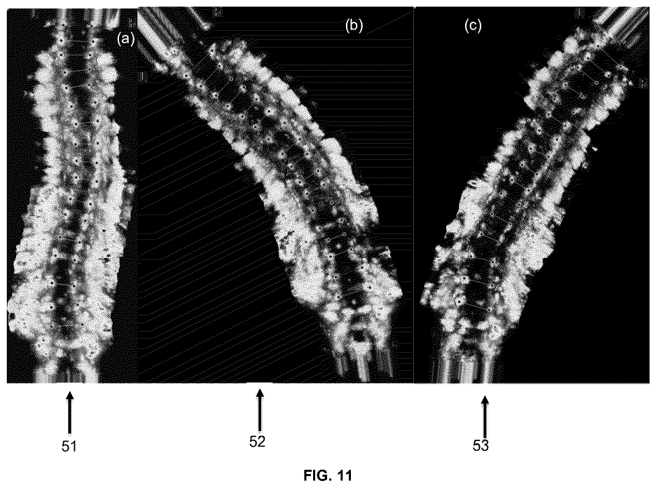

FIG. 11 shows the concept of the spine flexibility measurement. The coronal standing ultrasound image is shown as 51, in which the right thoracic Cobb angle is 27° and the left lumbar Cobb angle is 23°. The image labelled as 52 shows the prone ultrasound image with the patient bends to the left to reduce the left lumbar curve to 2°. The image labelled as 53 shows the prone ultrasound image with the patient bends to the right to reduce the right thoracic curve to 3°. The flexibility of the curve is (Standing Cobb−the Cobb angle at bending)/standing Cobb. For example, the left curve flexibility=(23−2)/23*100%=91.3%, which indicates very flexible.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the embodiments described herein.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc. can be passed, forwarded, or transmitted via any suitable means including memory sharing, message passing, token passing, network transmission, etc.

The actual software code or specialized control hardware used to implement these systems and methods is not limiting of the embodiments described herein. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

Although a few embodiments have been shown and described herein, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

### REFERENCES

1. Lonstein J. E.: Adolescent Idiopathic Scoliosis, The Lancet, 1994, 344:1407-1412.
2. Scholten P J M, Veldhuizen A G: Analysis of Cobb angle measurements in scoliosis, Clinical Biomechanics 1987, 2:7-13.
3. Drerup B: Improvements in measuring vertebral rotation from the projections of the pedicles. Journal of Biomechanics 1985, 18:369-378.
4. Perdriolle R, Vidal J: Thoracic idiopathic scoliosis curve evaluation and prognosis. Spine 1985, 10:785-791.
5. Nash C, Moe J H: A study of vertebral rotation. Journal of Bone and Joint Surgery 1969, 51:223-229.
6. Drerup B: Principles of measurement of vertebral rotation from frontal projection of the pedicles. Journal of Biomechanics 1984, 17:923-935.
7. Stokes I A F, Bigalow L C, Moreland M S: Measurement of axial rotation of vertebrae in scoliosis. Spine 1986, 11:213-218.
8. Lam GC: Vertebral rotation measurement: a summary and comparison of common radiographic and CT methods. Scoliosis 2008, 3:16.
9. Zheng R, Hill D, Hedden D, Mahood J, Moreau M, Southon S, Lou E: Assessment of Curve progression on Children with idiopathic scoliosis using Ultrasound Imaging method, European Spine, 27(9):2114-2119, doi:10.1007/s00586-017-5457-0, Jan. 10 2018.
10. Vo Q, Le L H, Lou E: A semi-automatic 3D ultrasound reconstruction method to assess the true severity of adolescent idiopathic scoliosis, Journal of Medical & Biological Engineering & Computing, doi:10.1007/s11517-019-02015-9, 57(10):2115-2118, 2019.
11. Sayed T, Khodaei M, Hill D, Lou E: Intra-and Inter-rater Reliabilities and Accuracy of Kyphotic Angle Measurements on Ultrasound Images versus Radiographs for Children with Adolescent Idiopathic Scoliosis-A Preliminary Study. Journal of Spine Deformity, doi:10.1007/s43390-021-00466-5, 10(3): 501-507, 2022.
12. Wong J, Parent E, Reformat M, Lou E: Validity and Accuracy of Automatic Cobb Angle Measurement on 3D Spinal Ultrasonographs for Children with Adolescent Idiopathic Scoliosis SOSORT 2024 Award Winner, European Spine Journal, https://doi.org/10.1007/s00586-024-08376-6, July 2024.
13. Nguyen T N N, Le L H, Emery D, Stampe K, Southon Hryniuk S, Lou E: Reliability and Accuracy of Scoliotic Parameters on Using a Wireless Handheld 3D Ultrasound for Children with Adolescent Idiopathic Scoliosis: A Pilot Study, European Spine, https://doi.org/10.1007/s00586-024-08445-w, 2024.
14. Lai K K L, Lee T T Y, Lee M K S, Hui J C H, Zheng Y P: Validation of Scolioscan Air-Portable Radiation Free Three-Dimensional Ultrasound Imaging Assessment System for Scoliosis, Sensor, doi:10.3390/s21082858, 21(8), 2858, 2021.

### Patents on Using Ultrasound for Scoliosis

200810094381.9 (China)
U.S. Pat. No. 8,900,146 B2 (USA)
ZL201080040696.0 (China)
5849048 (Japan)
2,769,150 (Canada)
2010278526 (Australia)

We claim:

1. An imaging system for screening, diagnosing, monitoring, and assessing treatment of a scoliotic patient, the system comprising:
a) an ultrasound scanner for capturing internal spinal structural changes of the patient by acquiring a stream of axial B-mode images thereof (“ultrasound images”);
b) a stereo-camera to capture images of a back surface topography of the patient (“stereo-camera images”);
c) an inertia measurement unit (“IMU”) disposed inside the stereo-camera to produce position and orientation information of the stereo-camera (“IMU data”);
d) a mini-personal computer (“PC”) operatively coupled to the stereo-camera for storing the IMU data and for storing the stereo-camera images;
e) a battery for providing electrical power for the mini-PC and for the stereo-camera;
f) a central processing unit comprising a display, and at least one software module comprising software code segments configured for:
i) reconstructing at least one spine and back surface image from the ultrasound images and the stereo-camera images,
ii) displaying and manipulating the at least one spine and back surface image in three dimensions (“3D”),
iii) manually annotating spinal and surface landmarks on the at least one spine and back surface image,
iv) automatically annotating the spinal and surface landmarks using machine learning,
v) measuring scoliotic and surface topography parameters based on the annotated spinal and surface landmarks, and
vi) predicting curve progression using machine learning; and
g) a wireless control pedal for controlling the operation of the system.

2. The system as set forth in claim 1, further configured to wirelessly stream the ultrasound images to the central processing unit with a time stamp indicated on each image thereof.

3. The system as set forth in claim 1, wherein the mini-PC is configured to transfer the IMU data and the stereo-camera images with at least one time stamp associated with each thereof to the central processing unit after an ultrasound scan of the patient has been completed.

4. The system as set forth in claim 3, wherein the at least one software module is configured to synchronize the IMU data and the stereo-camera images at the at least one time stamp.

5. The system as set forth in claim 4, wherein the software code segments are further configured to align the ultrasound images with the IMU data and the stereo-camera images at the at least one time stamp.

6. The system as set forth in claim 1, wherein the software code segments are further configured to reconstruct the at least one spine and back surface image in 3D based on the IMU data and the stereo-camera images.

7. The system as set forth in claim 1, wherein the system is configured to synchronize the ultrasound data images with the stereo-camera images.

8. The system as set forth in claim 1, wherein the system is configured to calculate position and orientation data of the ultrasound scanner transducer using the IMU data and stereo-camera images.

9. A method for screening, diagnosing, monitoring, and assessing treatment of a scoliotic patient, comprising using an imaging system, comprising:
a) an ultrasound scanner for capturing internal spinal structural changes of the patient by acquiring a stream of axial B-mode images thereof (“ultrasound images”);
b) a stereo-camera to capture images of a back surface topography of the patient (“stereo-camera images”);
c) an inertia measurement unit (“IMU”) disposed inside the stereo-camera to produce position and orientation information of the stereo-camera (“IMU data”);
d) a mini-personal computer (“PC”) operatively coupled to the stereo-camera for storing the IMU data and for storing the stereo-camera images;
e) a battery for providing electrical power for the mini-PC and for the stereo-camera;
f) a central processing unit comprising a display, and at least one software module comprising software code segments configured for:
i) reconstructing at least one spine and back surface image from the ultrasound images and the stereo-camera images,
ii) displaying and manipulating the at least one spine and back surface image in three dimensions (“3D”),
iii) manually annotating spinal and surface landmarks on the at least one spine and back surface image,
iv) automatically annotating the spinal and surface landmarks using machine learning,
v) measuring scoliotic and surface topography parameters based on the annotated spinal and surface landmarks, and
vi) predicting curve progression using machine learning; and
g) a wireless control pedal for controlling the operation of the system.

10. The method as set forth in claim 9, further comprising wirelessly streaming the ultrasound images to the central processing unit with a time stamp indicated on each image thereof.

11. The method as set forth in claim 9, further comprising transferring the IMU data and the stereo-camera images with at least one time stamp associated with each thereof to the central processing unit after an ultrasound scan of the patient has been completed.

12. The method as set forth in claim 11, further comprising synchronizing the IMU data and the stereo-camera images at the at least one time stamp.

13. The method as set forth in claim 12, further comprising aligning the ultrasound images with the IMU data and the stereo-camera images at the at least one time stamp.

14. The method as set forth in claim 9, further comprising reconstructing the at least one spine and back surface image in 3D based on the IMU data and the stereo-camera images.

15. The method as set forth in claim 9, further comprising synchronizing the ultrasound data images with the stereo-camera images.

16. The method as set forth in claim 9, further comprising calculating position and orientation data of the ultrasound scanner transducer using the IMU data and stereo-camera images.
